(19) United States
(12) Patent Application Publication
(10) Pub. No.: US 2012/0108973 A1
(43) Pub. Date: May 3, 2012

(54) ULTRASONIC DIAGNOSTIC APPARATUS AND ULTRASONIC IMAGE PROCESSING APPARATUS

(75) Inventor: Ryota Osumi, Nasushiobara-shi (JP)

(73) Assignees: TOSHIBA MEDICAL SYSTEMS CORPORATION, OTAWARA-SHI (JP); KABUSHIKI KAISHA TOSHIBA, TOKYO (JP)

(21) Appl. No.: 13/333,376

(22) Filed: Dec. 21, 2011

Related U.S. Application Data
(63) Continuation of application No. PCT/JP2011/075054, filed on Oct. 31, 2011.

(30) Foreign Application Priority Data
Nov. 1, 2010 (JP) 2010-245266

(51) Int. Cl. A61B 8/14; G06K 9/00

(52) U.S. Cl. 600/443; 382/131

(57) ABSTRACT

According to one embodiment, an edge information calculation unit calculates edge information based on a generated ultrasonic image. An edge filter unit generates a filtered image from the ultrasonic image by applying a filter having filter characteristics corresponding to the calculated edge information to the ultrasonic image. An edge enhancement unit generates an enhanced image from the filtered image by increasing the brightness value, of the filtered image, which corresponds to the edge information. A high brightness suppression unit generates a composite image of the enhanced image and the ultrasonic image in accordance with a compositing ratio corresponding to the brightness value of the enhanced image.

### CROSS REFERENCE TO RELATED APPLICATIONS

This application is a Continuation Application of PCT Application No. PCT/JP2011/075054, filed Oct. 31, 2011 and based upon and claiming the benefit of priority from prior Japanese Patent Application No. 2010-245266, filed Nov. 1, 2010, the entire contents of all of which are incorporated herein by reference.

### FIELD

Embodiments described herein relate generally to an ultrasonic diagnostic apparatus and an ultrasonic image processing apparatus.

### BACKGROUND

An ultrasonic diagnostic apparatus transmits ultrasonic waves from the transducers incorporated in an ultrasonic probe to a subject, receives the ultrasonic waves reflected by the subject via the transducers, and generates an ultrasonic image based on echo signals corresponding to the received ultrasonic waves. An ultrasonic image includes various kinds of noise and speckles due to the interference of ultrasonic waves in addition to information associated with the tissue of the subject. Noise and speckles degrade the image quality of ultrasonic images.

There is available a method of calculating the edge information of each pixel of an ultrasonic image and applying a filter corresponding to the calculated edge information to each pixel in order to reduce noise and speckles and enhance information associated with the tissue of a subject. More specifically, this filter smoothes information in the edge direction and sharpens information in a direction perpendicular to the edge direction. An image processing method using the filter is used to, for example, improve the image quality of a blood vessel image.

In order to observe an ultrasonic image associated with a blood vessel, it is preferable to enhance the overall vascular wall intima region and perform smoothing in the intima direction without enhancing the parenchymal region located near the vascular wall intima region. Although the above image processing method detects a vascular wall intima region as an edge, it also detects a parenchymal region exhibiting a large brightness change as an edge. Therefore, enhancing a vascular wall intima region will also enhance a parenchymal region. As described above, when optimizing the display of a vascular wall intima region by using the above image processing method, it may excessively increase the brightness of the parenchymal region near the vascular wall intima region.

It is an object of an embodiment to provide an ultrasonic diagnostic apparatus and ultrasonic image processing apparatus which can improve the image quality of ultrasonic images.

### BRIEF DESCRIPTION OF THE DRAWINGS

FIG. 1 is a block diagram showing the arrangement of an ultrasonic diagnostic apparatus according to an embodiment.

FIG. 2 is a block diagram showing the arrangement of an image processing unit in FIG. 1.

FIG. 3 is a block diagram showing the arrangement of an optimal brightness image generation unit in FIG. 2.

FIG. 4 is a block diagram showing the arrangement of a high brightness suppression unit in FIG. 3.

FIG. 5 is a view showing an example of a blood vessel image as a Target image for the high brightness suppression unit in FIG. 3.

FIG. 6 is a graph showing the relationship between a parameter E_{TH} used by an image compositing unit in FIG. 5 and the brightness value of an enhanced image I_{ENH}.

FIG. 7 is a block diagram showing the arrangement of an optimal brightness image generation unit according to the first modification of this embodiment.

FIG. 8 is a block diagram showing the arrangement of an optimal brightness image generation unit according to the second modification of this embodiment.

FIG. 9 is a graph showing the input/output characteristic of an LOT used by a table unit in FIG. 8.

FIG. 10 is a block diagram showing the arrangement of an optimal brightness image generation unit according to the third modification of this embodiment.

### DETAILED DESCRIPTION

In general, according to one embodiment, an ultrasonic diagnostic apparatus includes an ultrasonic probe, a generation unit, a calculation unit, a filter processing unit, an enhancement unit, and a compositing unit. The ultrasonic probe transmits an ultrasonic wave to a subject, receives an ultrasonic wave reflected by the subject, and generates an echo signal corresponding to the received ultrasonic wave. The generation unit generates an ultrasonic image associated with the subject based on the generated echo signal. The calculation unit calculates edge information based on the generated ultrasonic image. The filter processing unit generates a filtered image from the ultrasonic image by applying a filter having a filter characteristic corresponding to the calculated edge information to the ultrasonic image. The enhancement unit generates an enhanced image from the generated filtered image by increasing a brightness value, of the filtered image, which corresponds to the edge information. The compositing unit generates a composite image of the generated enhanced image and the ultrasonic image in accordance with a compositing ratio corresponding to a brightness value of the enhanced image.

An ultrasonic diagnostic apparatus and image processing apparatus according to an embodiment will be described below with reference to the accompanying drawings.

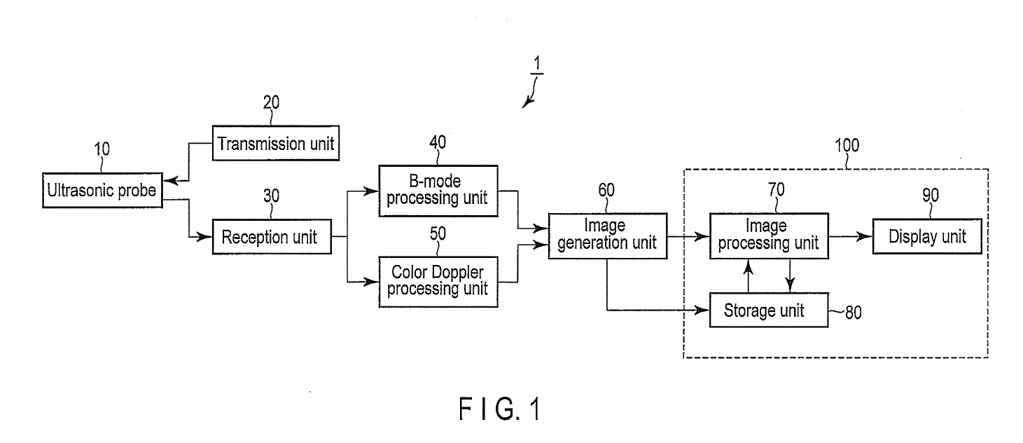

FIG. 1 is a block diagram showing the arrangement of an ultrasonic diagnostic apparatus 1 according to this embodiment. The ultrasonic diagnostic apparatus 1 shown in FIG. 1 includes an ultrasonic probe 10, a transmission unit 20, a reception unit 30, a B-mode processing unit 40, a color Doppler processing unit 50, an image generation unit 60, an image processing unit 70, a storage unit 80, and a display unit 90.

The ultrasonic probe 10 includes a plurality of transducers. Upon receiving a driving signal from the transmission unit 20, the ultrasonic probe 10 transmits an ultrasonic wave to a subject. The ultrasonic wave transmitted to the subject is sequentially reflected by a discontinuity surface of acoustic impedance of internal body tissue. The ultrasonic probe 10 receives the reflected ultrasonic wave. The ultrasonic probe 10 generates an electrical signal (echo signal) corresponding to the intensity of the received ultrasonic wave. The amplitude of the echo signal depends on an acoustic impedance difference on the discontinuity surface by which the echo signal is reflected. When an ultrasonic wave is reflected by the surface of a moving subject such as a moving blood flow or a cardiac wall, the echo signal is subjected to a frequency shift depending on the velocity component of the moving subject in the ultrasonic transmission direction due to a Doppler effect.

The transmission unit 20 repeatedly transmits ultrasonic waves to a subject via the ultrasonic probe 10. More specifically, the ultrasonic transmission unit 20 includes a rate pulse generation circuit, transmission delay circuit, and driving pulse generation circuit (none of which are shown) for the transmission of ultrasonic waves. The rate pulse generation circuit repeatedly generates rate pulses for each channel at a predetermined rate frequency fr Hz (period: 1/fr sec). The delay circuit gives each rate pulse the delay time required to focus an ultrasonic wave into a beam and determine transmission directivity for each channel. The driving pulse generation circuit applies a driving pulse to the ultrasonic probe 10 at the timing based on each delayed rate pulse.

The reception unit 30 repeatedly receives transmission waves from the subject via the ultrasonic probe 10. More specifically, the reception unit 30 includes an amplifier circuit, A/D converter, reception delay circuit, and adder (none of which are shown) for the reception of ultrasonic waves. The amplifier circuit amplifies echo signals from the ultrasonic probe 10 on a channel basis. The A/D converter converts the amplified echo signals from analog signals to digital signals on a channel basis. The reception delay circuit gives each echo signal converted into a digital signal the delay time required to focus the signal into a beam and determine reception directivity for each channel. The adder then adds the respective echo signals to which the delay times are given. With this addition processing, reception signals corresponding to reception beams are generated. In this manner, the reception unit 30 generates a plurality of reception signals respectively corresponding to a plurality of reception beams. The reception signals are supplied to the B-mode processing unit 40 and the color Doppler processing unit 50.

The B-mode processing unit 40 logarithmically amplifies reception signals from the reception unit 30 and detects the envelopes of the logarithmically amplified reception signals, thereby generating the data of B-mode signals representing the intensities of the echo signals by brightness. The data of the generated B-mode signals are supplied to the image generation unit 60.

The color Doppler processing unit 50 performs autocorrelation processing for reception signals from the reception unit 30 to extract a blood flow, tissue, and contrast medium echo component by the Doppler effect, and generates the data of a Doppler signal expressing the intensity of blood flow information such as an average velocity, variance, and power in color. The generated data of the Doppler signal is supplied to the image generation unit 60.

The image generation unit 60 generates a B-mode image associated with the subject based on B-mode signals from the B-mode processing unit 40. More specifically, the image generation unit 60 is formed from a scan converter. The image generation unit 60 generates a B-mode image by converting the scan scheme of a B-mode signal from the ultrasonic scan scheme to the display device scheme. The pixels of a B-mode image have brightness values corresponding to the intensities of B-mode signals from which they originate. Likewise, the image generation unit 60 generates a Doppler image associated with the subject based on Doppler signals from the color Doppler processing unit 50. The pixels of the Doppler image have color values corresponding to the intensities of the Doppler signal from which they originate. The B-mode and Doppler image are supplied to the storage unit 80 and the image processing unit 70.

The image processing unit 70 executes image processing for the B-mode image from the image generation unit 60 or the image processing unit 70. This image processing generates a B-mode image in which speckles and noise are reduced and a region of interest is properly enhanced without excessively enhancing other regions. The details of the image processing will be described later. The B-mode image having undergone the image processing is supplied to the storage unit 80 and the display unit 90.

The display unit 90 displays, on the display device, the B-mode image processed by the image processing unit 70. In this case, a Doppler image may be superimposed on the B-mode image. It is possible to use, as a display unit, for example, a display device such as a CRT display, liquid crystal display, organic EL display, plasma display, or the like, as needed.

Note that the image processing unit 70, the storage unit 80, the display unit 90 constitute an image processing apparatus 100. As shown in FIG. 1, the image processing apparatus 100 may be incorporated in the ultrasonic diagnostic apparatus 1 or incorporated in a computer separate from the ultrasonic diagnostic apparatus 1.

The image processing unit 70 according to this embodiment will be described in detail below. Assume that a B-mode image to be processed by the image processing unit 70 is a B-mode image associated with a blood vessel of a subject. However, this embodiment is not limited to this, and it is possible to use a B-mode image associated with plastic tissue such as a bone or muscle other than blood vessels as a B-mode image to be processed by the image processing unit 70.

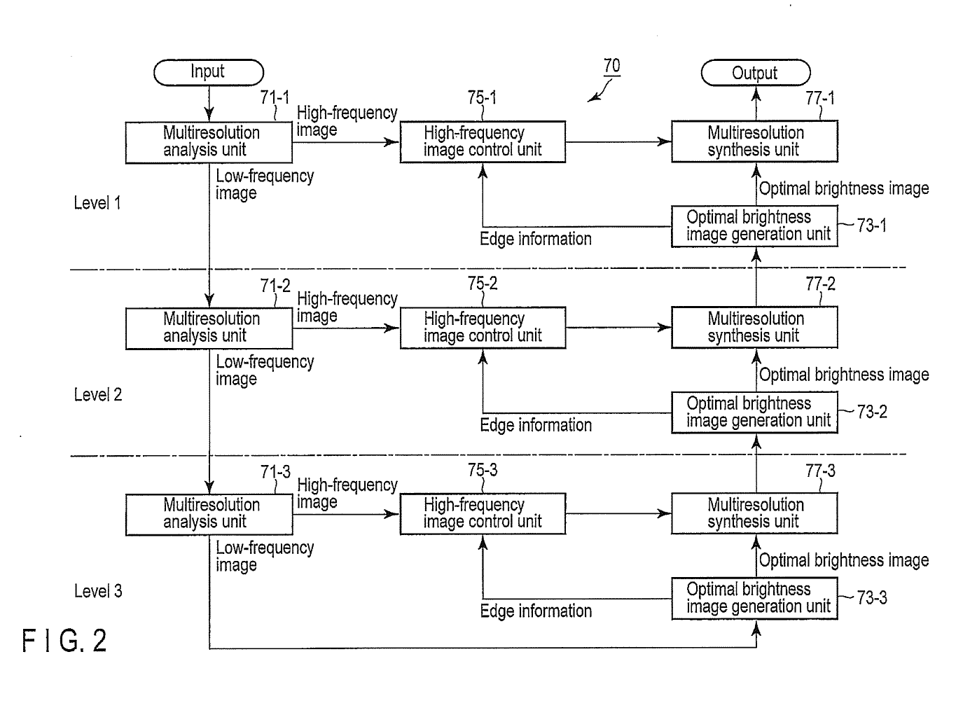

FIG. 2 is a block diagram showing the arrangement of the image processing unit 70. As shown in FIG. 2, the image processing unit 70 has a multiple structure constituted by a plurality of hierarchical layers (levels) for performing Multiresolution analysis/synthesis. For the sake of a concrete description, assume that the highest level of Multiresolution analysis/synthesis is 3. However, this embodiment need not be limited to this. It is possible to perform Multiresolution analysis/synthesis in the range from level 1 to level n (where n is a natural number equal to or more than 2). This embodiment uses discrete wavelet transform/inverse transform as an example of Multiresolution analysis/synthesis. However, the embodiment need not be limited to this. For example, it is possible to use, as Multiresolution analysis/synthesis, an existing Multiresolution analysis/synthesis method such as the Laplacian pyramid method or Gabor transform/inverse transform.

As shown in FIG. 2, the image processing unit 70 according to this embodiment includes Multiresolution analysis units 71 (71-1, 71-2, and 71-3), optimal brightness image generation units 73 (73-1, 73-2, and 73-3), high-frequency image control units 75 (75-1, 75-2, and 75-3), and Multiresolution synthesis units 77 (77-1, 77-2, and 77-3).

The Multiresolution analysis unit 71 generates a low-frequency image and a high-frequency image, each having a resolution lower than that of a Target image, based on the target image. For example, the Multiresolution analysis unit 71 performs discrete wavelet transform for the target image. In discrete wavelet transform, the Multiresolution analysis unit 71 applies each of one-dimensional low-frequency and high-frequency filters in each axis direction (each dimension) of xy orthogonal coordinates. Applying these filters to the target image will decompose it into one low-frequency image and three high-frequency images. The low-frequency image includes low-frequency components of the spatial frequency components of the target image. Each high-frequency image includes high-frequency components, of the spatial frequency components of the target image, which are associated with at least one direction. The number of samples of each image after decomposition per each coordinate axis is half the number of samples per each coordinate axis before decomposition.

When the Multiresolution analysis unit 71 belongs to the lowest level (level 1 in the case in FIG. 2), the target image is a B-mode image from the image generation unit 60 or the image processing unit 70. When the Multiresolution analysis unit 71 does not belong to the lowest level (level 1 in the case in FIG. 2), the target image is a low-frequency image from the Multiresolution analysis unit 71 at the immediately lower level.

When the Multiresolution analysis unit 71 belongs to the highest level (level 3 in the case in FIG. 2), the generated low-frequency image is supplied to the optimal brightness image generation unit 73-3 at the highest level. When the Multiresolution analysis unit 71 does not belong to the highest level, the generated low-frequency image is supplied to the Multiresolution analysis unit 71 at the immediately higher level. The generated three high-frequency images are supplied to the high-frequency image control units 75 belonging to the same level.

The optimal brightness image generation unit 73 calculates the edge information of each of a plurality of pixels included in the target image. The edge information is supplied to the high-frequency image control unit 75 at the same level. The optimal brightness image generation unit 73 also generates an image in which speckles and noise are reduced and an edge region of a non-high-brightness region is properly enhanced without excessively enhancing a high-brightness region, from the target image by using the edge information. The generated image will be referred to as an optimal brightness image. The optimal brightness image is supplied to the Multiresolution synthesis unit 77 at the same level.

When the optimal brightness image generation unit 73 belongs to the highest level (level 3 in the case in FIG. 2), the target image is a low-frequency image from the Multiresolution analysis unit 71 belonging to the same highest level. When the optimal brightness image generation unit 73 does not belong to the highest level, the target image is an image from the Multiresolution synthesis unit 77 belong to the immediately higher level.

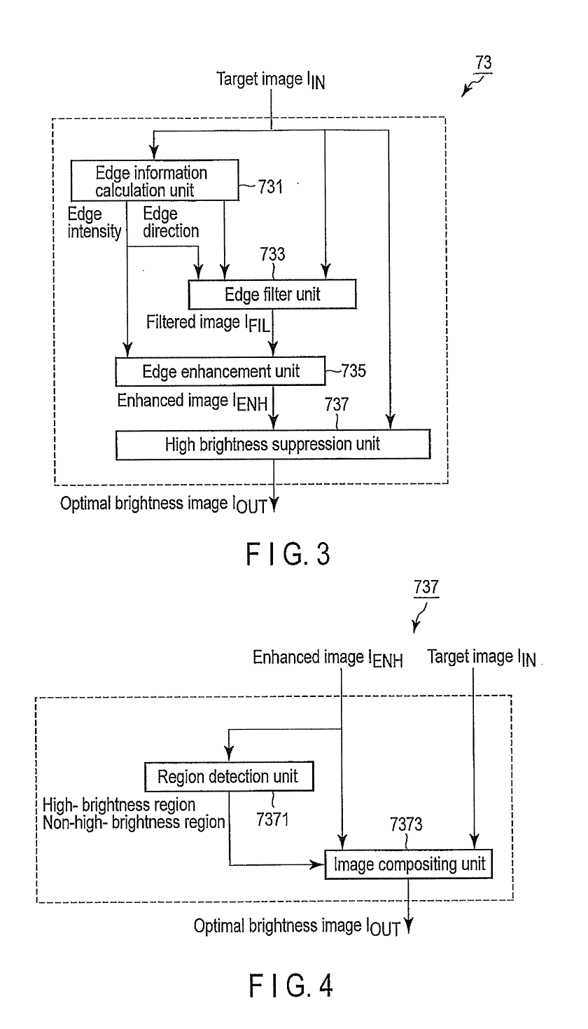

FIG. 3 is a block diagram showing the arrangement of the optimal brightness image generation unit 73. As shown in FIG. 3, the optimal brightness image generation unit 73 includes an edge information calculation unit 731, an edge filter unit 733, an edge enhancement unit 735, and a high brightness suppression unit 737.

The edge information calculation unit 731 calculates the edge information of each of a plurality of pixels included in a Target image T_{IN}. More specifically, first of all, the edge information calculation unit 731 calculates a spatial derivative value by performing spatial derivation along each coordinate axis using a processing target pixel and its neighboring pixels. The edge information calculation unit 731 then calculates the intensity and direction of an edge associated with the processing target pixel based on the calculated spatial derivative value. The combination of the intensity and direction of this edge is edge information. More specifically, the edge information calculation unit 731 calculates a plurality of elements of the structure tensor of the processing target pixel by using the spatial derivative value. The edge information calculation unit 731 performs linear algebra operation for the plurality of calculated elements to calculate the two eigen values and two eigen vectors of the structure tensor. One of the two eigen vectors indicates a direction along the edge, and the other indicates a direction perpendicular to the edge. In this case, the direction along the edge will be referred to as an edge direction. An eigen value depends on the intensity of an edge.

Pixels whose edge information is to be calculated may be all the pixels included in the target image I_{IN} or pixels in the region of interest set by the user via an input device or the like. In addition, it is possible to calculate one piece of edge information for one pixel or a plurality of pixels. When one piece of edge information is to be calculated for a plurality of pixels, for example, the calculation may be performed for representative pixels of the plurality of pixels. A representative pixel is, for example, a pixel at the center, the barycenter, or an edge of a plurality of pixels. It is also possible to use the statistical value of a plurality of pieces of edge information of a plurality of pixels as the edge information of the plurality of pixels. In this case, the statistical value is set to, for example, the average value, median value, maximum value, minimum value, mode value, or the like of a plurality of pieces of edge information.

Note that a method of calculating edge information is not limited to the method using a structure tensor. For example, it is possible to calculate edge information by using a Hessian matrix in place of a structure tensor.

The edge filter unit 733 applies a filter having filter characteristics corresponding to edge information to an input image. In this case, a filter having filter characteristics corresponding to edge information will be referred to as an edge filter. More specifically, the edge filter unit 733 calculates an edge filter for each of a plurality of pixels included in the target image I_{IN}. An edge filter has characteristics to sharpen an edge region along the edge direction and smooth an edge region along a direction perpendicular to the edge direction. An edge filter includes, for example, a nonlinear anisotropic diffusion filter calculated based on edge information. The edge filter unit 733 applies an edge filter to each pixel to enhance an edge region included in the target image I_{IN} and suppresses a non-edge region. In this case, an output image from the edge filter unit 733 will be referred to as a filtered image I_{FIL}.

The edge enhancement unit 735 increases the brightness value of each of a plurality of pixels included in the filtered image I_{FIL} in accordance with edge information. In this case, an output image from the edge enhancement unit 735 will be referred to as an enhanced image I_{ENH}. More specifically, the edge enhancement unit 735 compares the edge intensity of each pixel with a threshold. The edge enhancement unit 735 sets each pixel having edge intensity higher than the threshold to an edge region, and sets each pixel having an edge intensity lower than the threshold to a non-edge region. The edge enhancement unit 735 then increases the brightness values of the pixels included in the edge region by increase amounts corresponding to the edge intensities. An increase amount is defined by, for example, the product of a parameter a_{ENH} and an edge intensity E_{EDGE}. The enhancement of an edge region is expressed by, for example, equation (1) given below. Note that I_{ENH} represents the brightness value of a pixel of an enhanced image, and I_{FIL} represents the brightness value of a pixel of a filtered image.

I_{ENH}=I_{FIL}+(1+a_{ENH}·E_{EDGE}) (1)

The parameter a_{ENH} is a parameter for adjusting the degree of increase in brightness value. The operator arbitrarily sets the parameter a_{ENH}. Note that in order to avoid excessive enhancement, of an edge region, the parameter a_{ENH} is set to an amount as small as about 0.02. In this manner, the edge enhancement unit 735 further enhances an edge region on the filtered image I_{FIL} by slightly increasing the brightness values of pixels exhibiting relatively high edge intensities.

In this manner, the edge enhancement unit 735 increases the brightness value of a pixel corresponding to edge information. Note that when one piece of edge information is calculated for a plurality of pixels, the edge enhancement unit 735 increases the brightness values of the plurality of pixels corresponding to the edge information.

The high brightness suppression unit 737 suppresses a high-brightness region on the enhanced image I_{ENH} to generate an optimal brightness image. More specifically, the high brightness suppression unit 737 generates an optimal brightness image I_{OUT} by compositing the enhanced image I_{ENH} and the target image I_{IN} in accordance with a compositing ratio corresponding to the brightness value of the enhanced image I_{ENH}.

FIG. 4 is a block diagram showing the arrangement of the high brightness suppression unit 737. As shown in FIG. 4, the high brightness suppression unit 737 includes a region detection unit 7371 and an image compositing unit 7373. For the sake of a concrete description, the following will exemplify a B-mode image associated with a blood vessel (to be referred to as a blood vessel image hereinafter) as a Target image.

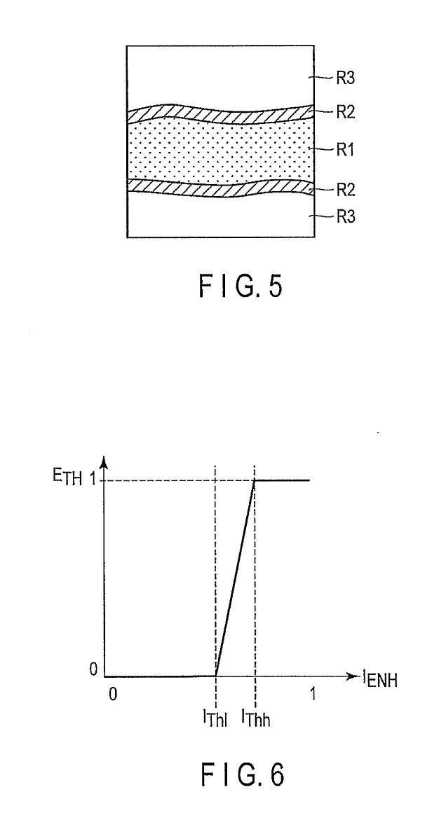

FIG. 5 is a view showing an example of a blood vessel image. As shown in FIG. 5, a blood vessel image includes a luminal region R1 associated with the lumen, a vascular wall intima region R2 associated with the vascular wall intima, and a parenchymal region R3 associated with the parenchymal region. Assume that a pixel region which the operator wants to observe thoroughly is the vascular wall intima region R2. The vascular wall intima region R2 is located between the luminal region R1 and the parenchymal region R3. If the vascular wall intima is normal, the vascular wall intima region R2 has a brightness value smaller than that of the parenchymal region R3. In general, the vascular wall intima region R2 is displayed in light gray on a B-mode image. The vascular wall intima region R2 has an elongated shape. Therefore, the vascular wall intima region R2 is recognized as an edge region by image processing. The edge filter unit 733 therefore enhances the vascular wall intima region R2. As described above, the edge filter unit 733 executes edge filter processing at each level of Multiresolution analysis. Since the resolution of the image is decreased by Multiresolution analysis, the vascular wall intima region R2 as an edge region is not satisfactorily reproduced on the image. For example, the vascular wall intima region R2, which is actually a one connected pixel region, is displayed as a plurality of segmented pixel regions due to a decrease in resolution. That is, it is not possible to satisfactorily enhance the vascular wall intima region R2 as an edge region of a non-high-brightness region by only sharpening operation using the edge filter unit 733. For this reason, the edge enhancement unit 735 on the subsequent stage of the edge filter unit 733 further enhances the vascular wall intima region R2 (the edge region of the non-high-brightness region). However, the edge enhancement unit 735 also further enhances the edge region of the high-brightness region by edge enhancement. As a consequence, the parenchymal region R3 on the enhanced image is excessively enhanced, resulting in excessive whiteness on the image.

The region detection unit 7371 detects a high-brightness region and a non-high-brightness region from the enhanced image I_{ENH}. More specifically, the region detection unit 7371 compares the brightness value of each of a plurality of pixels included in the enhanced image I_{ENH} with a threshold. If the brightness value of the processing target pixel is larger than the threshold, the region detection unit 7371 sets the processing target pixel as a high-brightness pixel. If the brightness value of the processing target pixel is smaller than the threshold, the region detection unit 7371 sets the processing target pixel as a non-high-brightness pixel. A set of high-brightness pixels is a high-brightness region, and a set of non-high-brightness pixels is a non-high-brightness region. The 7371 detects a high-brightness region and a non-high-brightness region from the enhanced image I_{ENH} by repeatedly comparing a brightness value with the threshold in this manner. When a vascular wall intima region is to be observed, a threshold for region detection is set to, for example, the maximum brightness value which the vascular wall intima region after enhancement can have so as to include the vascular wall intima region in a non-high-brightness region.

The image compositing unit 7373 generates an optimal brightness image in which an edge region of a high-brightness region is suppressed, and an edge region of a non-high-brightness region is enhanced. In terms of image processing, the image compositing unit 7373 generates the optimal brightness image I_{OUT} by compositing the enhanced image I_{ENH} and the target image I_{IN} in accordance with the compositing ratio between the enhanced image I_{ENH} and the target image I_{IN}. The compositing ratio indicates the ratio between the degree of contribution of the enhanced image I_{ENH} to the brightness value of the optimal brightness image and that of the target image I_{IN}. More specifically, a compositing ratio is determined in accordance with the brightness value of each of a plurality of pixels included in the enhanced image I_{ENH}. For example, a compositing ratio is set to the ratio of a weight coefficient for the target image I_{IN} to the total value of a weight coefficient for the enhanced image I_{ENH} and a weight coefficient for the target image I_{IN}. The total value of a weight coefficient for the enhanced image I_{ENH} and a weight coefficient for the target image I_{IN} is set to 1. A compositing ratio is set to, for example, a value which enhances a non-high-brightness region and suppresses a high-brightness region. There are two types of compositing ratios according to this embodiment. These two types of compositing ratios will be described below.

The first compositing ratio: when a processing target pixel is sorted to a high-brightness region, the compositing ratio is 100%, that is, the weight coefficient for the enhanced image I_{ENH} is set to 0, and the weight coefficient for the target image I_{IN} is set to 1. When a processing target pixel is sorted to a non-high-brightness region, the compositing ratio is 0%, that is, the weight coefficient for the enhanced image I_{ENH} is set to 1, and the weight coefficient for the target image I_{IN} is set to 0. That is, the image compositing unit 7373 replaces the brightness value of a high-brightness pixel included in the enhanced image I_{ENH} with the brightness value of a pixel at the same coordinates of the target image I_{IN}. In other words, the image compositing unit 7373 selects the enhanced image I_{ENH} in a high-brightness region, and the target image I_{IN} in a non-high-brightness region. Therefore, the image compositing unit 7373 can generate an optimal brightness image in which the vascular wall intima region is further enhanced and the parenchymal region is properly suppressed, by using the first compositing ratio based on the target image I_{IN} and the enhanced image I_{ENH}.

The second compositing ratio: for example, equation (2) given below expresses the processing of generating an optimal brightness image by using the second compositing ratio. Note that I_{OUT} represents the brightness value of a pixel of an optimal brightness image, I_{IN} represents the brightness value of a pixel of a Target image, and I_{ENH} represents the brightness value of a pixel of an enhanced image.

I_{OUT}=E_{TH}·I_{IN}+(1−E_{TH})·I_{ENH} (2)

A parameter E_{TH} is a weight coefficient for the target image I_{IN}, and (1−E_{TH}) is a weight coefficient for the enhanced image I_{ENH}.

FIG. 6 is a graph showing the relationship between the parameter E_{TH} and the brightness value of the enhanced image I_{ENH}. As shown in FIG. 6, the weight coefficient E_{TH} for the target image I_{IN} linearly changes in accordance with the brightness values of the enhanced image I_{ENH} to smooth the boundary between a high-brightness region and the non-high-brightness region. More specifically, when a processing target pixel is sorted to a non-high-brightness region, the weight coefficient E_{TH} is set to 0, and the weight coefficient (1−E_{TH}) is set to 1. That is, when a processing target pixel is sorted to a non-high-brightness region, the compositing ratio is set to 0%. When a processing target pixel is set to a high-brightness region, the weight coefficient E_{TH} increases from 0 to 1, and the weight coefficient (1−E_{TH}) linearly decreases from 1 to 0 as the brightness value of the processing target pixel increases. That is, when a processing target pixel is sorted to a high-brightness region, the compositing ratio is linearly changed from 0% to 100% with an increase in brightness value. More specifically, as the brightness value increases from a threshold I_{THl} to a threshold I_{Thh}, the weight coefficient E_{TH} linearly decreases from 0 to 1. The threshold I_{THl} is set, for example, between the maximum brightness value which a vascular wall intima region can have and the minimum brightness value which the parenchymal region can have. The threshold I_{Thh} is set, for example, to a value larger than the minimum brightness value which the parenchymal region can have by a specified value.

As described above, the second compositing ratio linearly changes in accordance with the brightness value in the brightness value range which a high-brightness region can take. This allows the image compositing unit 7373 to smooth the boundary between a high-brightness region and a non-high-brightness region on an optimal brightness image as compared with the case using the first compositing ratio. Therefore, the image compositing unit 7373 can generate the optimal brightness image I_{OUT} in which the vascular wall intima region is further enhanced, and the parenchymal region is properly suppressed, by using the second compositing ratio based on the target image I_{IN} and the enhanced image I_{ENH}.

The operator can arbitrarily set the first or second compositing ratio to be used. The optimal brightness image I_{OUT} generated by using the first or second compositing ratio in this manner is supplied to the Multiresolution synthesis unit 77 at the same level.

Processing on the subsequent stage of the optimal brightness image generation unit 73 will be described next by referring back to FIG. 2.

The high-frequency image control unit 75 controls the brightness values of three high-frequency images from the Multiresolution analysis unit 71 by using edge information from the optimal brightness image generation unit 73. More specifically, the high-frequency image control unit 75 multiplies each of a plurality of pixels included in each high-frequency image by a parameter corresponding to edge information. This parameter includes the first parameter for an edge region and the second parameter for a non-high-brightness region. The first parameter is set to enhance an edge region. The second parameter is set to suppress a non-high-brightness region. The high-frequency image whose brightness value is controlled by the high-frequency image control unit 75 is supplied to the Multiresolution synthesis unit 77.

Based on the optimal brightness image from the optimal brightness image generation unit 73 and the three high-frequency images from the high-frequency image control unit 75, the Multiresolution synthesis unit 77 generates an output image higher in resolution than the optimal brightness image or the high-frequency images. More specifically, the Multiresolution synthesis unit 77 performs Multiresolution synthesis such as discrete wavelet inverse transform for the optimal brightness image and the three high-frequency images. The number of samples per each coordinate axis of an output image after compositing operation is increased to twice the number of samples per each coordinate axis of an optimal brightness image or high-frequency image before compositing operation.

When the Multiresolution synthesis unit 77 does not belong to the lowest level (level 1 in the case in FIG. 2), an output image is supplied to the optimal brightness image generation unit 73 belonging to the immediately lower level. When the Multiresolution synthesis unit 77 belongs to the lowest level, the image processing unit 70 supplies an output image to the display unit 90.

As described above, the ultrasonic diagnostic apparatus 1 and the image processing apparatus 100 according to this embodiment include the edge filter units 733, the edge enhancement units 735, and the high brightness suppression units 737. The edge filter unit 733 applies an edge filter having filter characteristics corresponding to edge information to an input image. This generates a filtered image in which smoothing is performed in the edge direction, and sharpening is performed in a direction perpendicular to the edge direction. The edge enhancement unit 735 generates an enhanced image in which the brightness value of an edge region is further increased in accordance with the edge information. The high brightness suppression unit 737 suppresses a high-brightness region on the enhanced image. More specifically, the high brightness suppression unit 737 composites the enhanced image and the input image in accordance with a compositing ratio corresponding to the brightness value of the enhanced image. This allows the optimal brightness image generation unit 73 to generate an optimal brightness image in which speckles and noise are reduced and an edge region of a non-high-brightness region is properly enhanced without excessively enhancing a high-brightness region. More specifically, the optimal brightness image generation unit 73 can optimize the brightness value of the parenchymal region adjacent to the vascular wall intima region without excessively increasing the brightness value. The optimal brightness image generation unit 73 can also form the vascular wall intima region into one connected pixel region.

In this embodiment, the edge enhancement unit 735 performs edge enhancement, and the high brightness suppression unit 737 performs high brightness suppression at each level upon Multiresolution analysis. This makes the boundary between an edge region and a non-edge region or the boundary between a high-brightness region and a non-high-brightness region look more natural than that in a case in which the edge enhancement unit 735 and the high brightness suppression unit 737 respectively perform edge enhancement and high brightness suppression after Multiresolution synthesis at level 1.

The ultrasonic diagnostic apparatus 1 and image processing apparatus 100 according to this embodiment achieve an improvement in the image quality of an ultrasonic image in the above manner.

The optimal brightness image generation unit 73 according to this embodiment is provided for each level of Multiresolution analysis, and processes a low-frequency image at each level as a processing target. However, this embodiment is not limited to this. The optimal brightness image generation unit 73 may process a high-frequency image as a processing target instead of a low-frequency image. In addition, the optimal brightness image generation units 73 may be provided for only some of the levels in Multiresolution analysis. Furthermore, the optimal brightness image generation unit 73 may process an image before Multiresolution analysis or an image after Multiresolution analysis as a processing target.

### (First Modification)

The optimal brightness image generation unit 73 according to this embodiment causes the high brightness suppression unit 737 to perform high brightness suppression after edge enhancement by the edge enhancement unit 735. An optimal brightness image generation unit according to the first modification is provided with an edge enhancement unit after the high brightness suppression unit 737. The optimal brightness image generation unit according to the first modification will be described below. Note that the same reference numerals in the following description denote constituent elements having almost the same functions, and a repetitive description will be made only when required.

FIG. 7 is a block diagram showing the arrangement of an optimal brightness image generation unit 73a according to the first modification. As shown in FIG. 7, the optimal brightness image generation unit 73a includes the edge information calculation unit 731, the edge filter unit 733, a high brightness suppression unit 737a, and an edge enhancement unit 735a.

The high brightness suppression unit 737a suppresses a high-brightness region on the filtered image I_{FIL} from the edge filter unit 733. More specifically, the high brightness suppression unit 737a generates a composite image I_{CON} by compositing the target image I_{IN} and the filtered image I_{FIL} in accordance with a compositing ratio corresponding to the brightness value of the filtered image I_{FIL}. A compositing ratio according to the first modification indicates the ratio between the degree of contribution of the filtered image I_{FIL} to the brightness value of the composite image I_{CON} and the degree of contribution of the target image I_{IN}. A compositing ratio according to the first modification is set to the ratio of a weight coefficient for the target image I_{IN} to the total value of a weight coefficient for the filtered image I_{FIL} and a weight coefficient for the target image I_{IN}. The composite image I_{CON} is an image in which a high-brightness region on the filtered image I_{FIL} is suppressed. This image compositing method is the same as that used by the image compositing unit 7373 in this embodiment, and hence a description of it will be omitted.

The edge enhancement unit 735a increases the brightness value of each of a plurality of pixels included in the composite image I_{CON} from the high brightness suppression unit 737a in accordance with edge information. This method of increasing a brightness value is the same as that used by the edge enhancement unit 735 according to this embodiment. The edge enhancement unit 735a generates an optimal brightness image in which an edge region of a high-brightness region is suppressed, and an edge region of a non-high-brightness region is enhanced. When performing ultrasonic examination on a blood vessel, this modification generates an optimal image in which the vascular wall intima region is further enhanced, and the parenchymal region is suppressed.

Therefore, an ultrasonic diagnostic apparatus and image processing apparatus according to the first modification of this embodiment achieve an improvement in the image quality of ultrasonic images in the above manner.

### (Second Modification)

The optimal brightness image generation unit 73 according to this embodiment is configured to generate an optimal brightness image based on a Target image and an enhanced image from the edge enhancement unit 735. An optimal brightness image generation unit according to the second modification generates an optimal brightness image based on only an enhanced image from the edge enhancement unit. The optimal brightness image generation unit according to the second modification will be described below. Note that the same reference numerals in the following description denote constituent elements having almost the same functions, and a repetitive description will be made only when required.

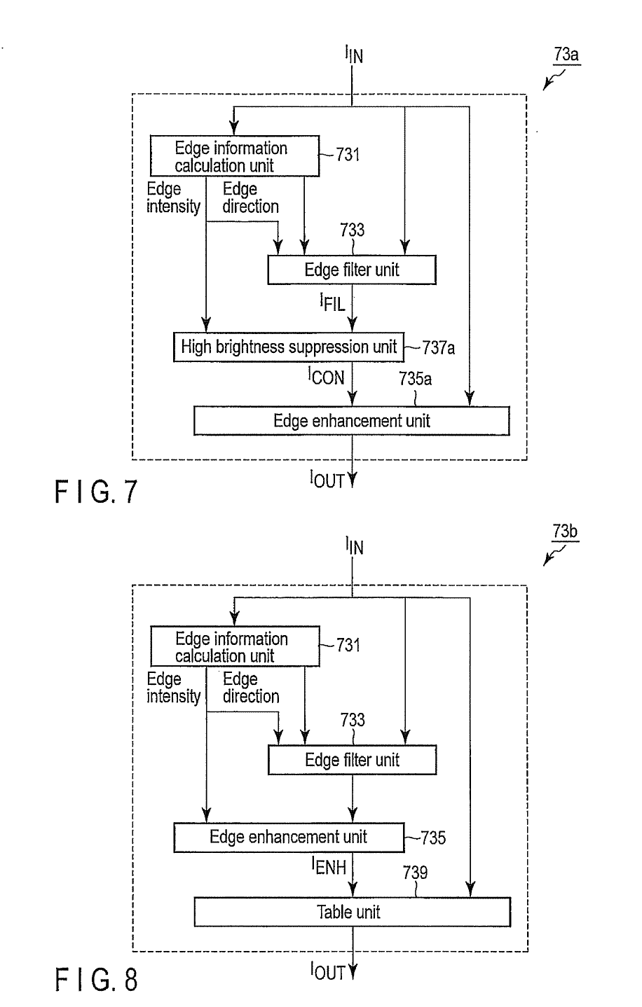

FIG. 8 is a block diagram showing the arrangement of the optimal brightness image generation unit 73b according to the second modification. As shown in FIG. 8, the optimal brightness image generation unit 73b includes the edge information calculation unit 731, the edge filter unit 733, the edge enhancement unit 735, and a table unit 739.

The table unit 739 applies an LUT (LookUp Table) to an enhanced image from the edge enhancement unit 735. Applying the LUT will generate the optimal brightness image I_{OUT}. The LOT is prepared in advance. The LUT is a table which specifies the input/output characteristics between input brightness values (the brightness values of the enhanced image I_{ENH}) and output brightness values (the brightness values of the optimal brightness image I_{OUT}).

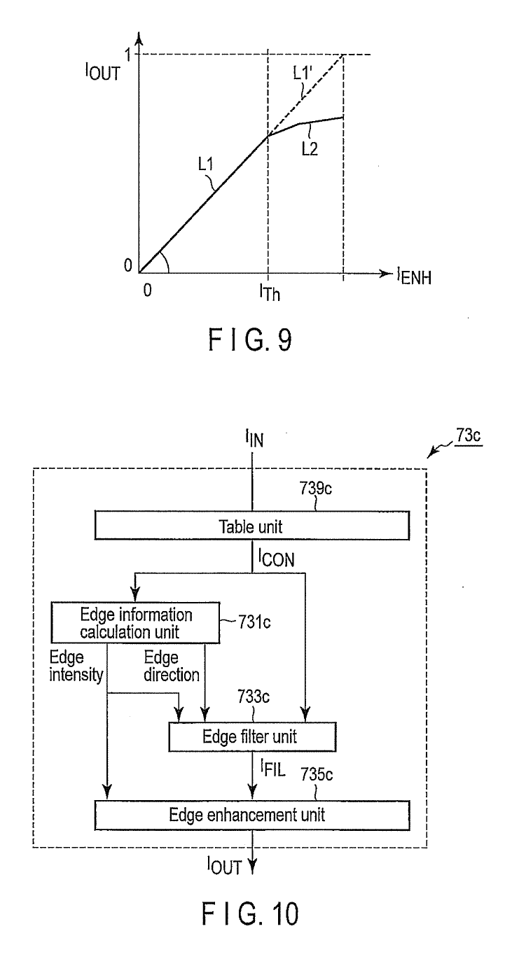

FIG. 9 is a graph showing the input/output characteristics of the LOT in the table unit 739. The abscissa represents input brightness values, and the ordinate represents output brightness values. As shown in FIG. 9, the LUT has the first and second input/output characteristics. The first input/output characteristic dominates a low brightness value range in which input brightness values are smaller than a threshold I_{TH}. In the first input/output characteristic, the output brightness value linearly increases with an increase in input brightness value. That is, a line L1 indicating output brightness values corresponding to input brightness values in the first input/output characteristic is a straight line, which has an inclination of 45° or more relative to the input brightness value axis. In this low brightness value range, for example, the value obtained by multiplying an input brightness value by a positive number equal to or more than 1 is replaced by an output brightness value. The second input/output characteristic dominates a high brightness value range in which input brightness values are larger than the threshold I_{Th}. In the second input/output characteristic, the output brightness value nonlinearly increases more gradually than in the first input/output characteristic with an increase in input brightness value. That is, a line L2 indicating output brightness values corresponding to input brightness values in the second input/output characteristic is a curved line, which nonlinearly rises below an extended line L1′ of the line L1. In this high brightness value range, for example, the value obtained by multiplying an input brightness value by a positive number less than 1 is replaced by an output brightness value. The threshold I_{Th} is set to the boundary between a high-brightness region and a non-high-brightness region. More specifically, the threshold I_{Th} is set to the maximum brightness value which a vascular wall intima region in an enhanced image can have, such that the vascular wall intima region is included in the non-high-brightness region.

Applying an LOT having such input/output characteristics to an enhanced image can generate an optimal brightness image in which the vascular wall intima region is further enhanced, and the parenchymal region is suppressed.

The ultrasonic diagnostic apparatus and image processing apparatus according to the second modification of this embodiment achieve an improvement in the image quality of ultrasonic images in the above manner.

### (Third Modification)

The optimal brightness image generation unit 73 according to this embodiment is provided with the high brightness suppression unit 737 on the subsequent stage of the edge filter unit 733. The optimal brightness image generation unit according to the third modification is provided with an edge filter unit on the subsequent stage of the high brightness suppression unit. The optimal brightness image generation unit according to third modification will be described below. Note that the same reference numerals in the following description denote constituent elements having almost the same functions as in this embodiment and the first and second modifications, and a repetitive description will be made only when required.

FIG. 10 is a block diagram showing the arrangement of an optimal brightness image generation unit 73c according to the third modification. As shown in FIG. 10, the optimal brightness image generation unit 73c includes a table unit 739c, an edge information calculation unit 731c, a main filter unit 733c, and an edge enhancement unit 735c.

The table unit 739c generates the table image I_{CON} by applying an LUT to the target image I_{IN}. The LUT has the same characteristics as the input/output characteristics according to the second modification.

The edge information calculation unit 731c calculates the edge information of each of a plurality of pixels included in the table image I_{CON}. The edge filter unit 733c applies an edge filter having filter characteristics corresponding to edge information to the table image I_{CON} to perform smoothing in the edge direction and sharpening in a direction perpendicular to the edge direction. This generates the filtered image I_{FIL}. The edge enhancement unit 735c increases the brightness value of each of a plurality of pixels included in the filtered image I_{FIL} in accordance with edge information. The method of increasing brightness values is the same as that used by the edge enhancement unit 735 according to this embodiment. The makes the edge enhancement unit 735c generate the optimal brightness image I_{OUT} in which, an edge region of a high-brightness region is properly suppressed, and an edge region of a non-high-brightness region is enhanced. When performing ultrasonic examination on a blood vessel, this modification generates an optimal image in which the vascular wall intima region is further enhanced, and the parenchymal region is suppressed.

Therefore, an ultrasonic diagnostic apparatus and image processing apparatus according to the third modification of this embodiment achieve an improvement in the image quality of ultrasonic images in the above manner.

Note that the image processing apparatus according to this embodiment described above processes an ultrasonic image as a processing target. However, the embodiment is not limited to this. That is, the image processing apparatus according to the embodiment can process images other than ultrasonic images, such as CT images generated by an X-ray computed tomography apparatus, X-ray images generated by an X-ray diagnostic apparatus, and MR images generated by a magnetic resonance imaging apparatus.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
an ultrasonic probe configured to transmit an ultrasonic wave to a subject, receive an ultrasonic wave reflected by the subject, and generate an echo signal corresponding to the received ultrasonic wave;
a generation unit configured to generate an ultrasonic image associated with the subject based on the generated echo signal;
a calculation unit configured to calculate edge information based on the generated ultrasonic image;
a filter processing unit configured to generate a filtered image from the ultrasonic image by applying a filter having a filter characteristic corresponding to the calculated edge information to the ultrasonic image;
an enhancement unit configured to generate an enhanced image from the generated filtered image by increasing a brightness value, of the filtered image, which corresponds to the edge information; and
a compositing unit configured to generate a composite image of the generated enhanced image and the ultrasonic image in accordance with a compositing ratio corresponding to a brightness value of the enhanced image.

2. The ultrasonic diagnostic apparatus of claim 1, wherein the calculation unit calculates edge information of each of a plurality of pixels included in the generated ultrasonic image based on a spatial distribution of brightness values of the respective pixels, and
the enhancement unit generates an enhanced image from the generated filtered image by increasing a brightness value of each of a plurality of pixels included in the filtered image in accordance with the edge information.

3. The ultrasonic diagnostic apparatus of claim 1, wherein a brightness value of a high-brightness region on the composite image is set to a brightness value of the ultrasonic image, and a brightness value of a non-high-brightness region on the composite image is set to a brightness value of the enhanced image.

4. The ultrasonic diagnostic apparatus of claim 1, wherein the compositing ratio is specified by a ratio between a first weight coefficient for the enhanced image and a second weight coefficient for the ultrasonic image, and
the compositing unit detects, from the enhanced image, a high-brightness region having a brightness value larger than a first threshold and a non-high-brightness region having a brightness value smaller than the first threshold, sets the first weight coefficient to 0 and the second weight coefficient to 1 in the high-brightness region, and sets the first weight coefficient to 1 and the second weight coefficient to 0 in the non-high-brightness region.

5. The ultrasonic diagnostic apparatus of claim 1, wherein the compositing ratio is specified by a ratio between a first weight coefficient for the enhanced image and a second weight coefficient for the ultrasonic image, and
the compositing unit
detects a high-brightness region and non-high-brightness region from the enhanced image, the high-brightness region having a brightness value larger than a first threshold, the non-high-brightness region having a brightness value smaller than the first threshold,
detects a first non-high-brightness region and a second non-high-brightness region from the non-high-brightness region, the first non-high-brightness region having a brightness value smaller than a second threshold smaller than the first threshold, the second non-high-brightness region having a brightness value larger than the second threshold,
sets the first weight coefficient to 1 and the second weight coefficient to 0 in the first non-high-brightness region, and
sets the first weight coefficient to a value from 1 to 0 and the second weight coefficient to a value from 0 to 1 in accordance with a brightness value in the second non-high-brightness region.

6. The ultrasonic diagnostic apparatus of claim 1, wherein the generation unit comprises an ultrasonic image generation unit configured to generate an original ultrasonic image associated with the subject based on the echo signal, and a Multiresolution analysis unit configured to generate a low-frequency image and a high-frequency image based on the generated original ultrasonic image, each of the low-frequency image and the high-frequency image having a resolution lower than a resolution of the ultrasonic image, and
the low-frequency image is used as the ultrasonic image.

7. The ultrasonic diagnostic apparatus of claim 6, further comprising a Multiresolution synthesis unit configured to generate an output image having the same resolution as that of the original ultrasonic image based on the composite image and the high-frequency image.

8. The ultrasonic diagnostic apparatus of claim 1, wherein the ultrasonic image is an image associated with a blood vessel of the subject.

9. The ultrasonic diagnostic apparatus of claim 1, wherein the enhancement unit changes a degree of increase in the brightness value in accordance with an edge intensity based on the edge information.

10. An ultrasonic diagnostic apparatus comprising:
an ultrasonic probe configured to transmit an ultrasonic wave to a subject, receive an ultrasonic wave reflected by the subject, and generate an echo signal corresponding to the received ultrasonic wave;
a generation unit configured to generate an ultrasonic image associated with the subject based on the generated echo signal;
a calculation unit configured to calculate edge information of each of a plurality of pixels included in the generated ultrasonic image based on spatial derivation of a brightness value of said each pixel;
a filter processing unit configured to generate a filtered image from the ultrasonic image by applying a filter having a filter characteristic corresponding to the calculated edge information to the ultrasonic image;
an enhancement unit configured to generate an enhanced image from the generated filtered image by increasing a brightness value of each of a plurality of pixels included in the filtered image in accordance with the edge information; and
a table application unit configured to generate a table image from the generated enhanced image by applying a table to the enhanced image, the table having a first input/output characteristic in which an output brightness value linearly increases with an increase in input brightness value in a brightness value range smaller than a threshold, and a second input/output characteristic in which an output brightness value nonlinearly increases more gradually than in the first input/output characteristic with an increase in input brightness value in a brightness value range larger than the threshold.

11. An ultrasonic diagnostic apparatus comprising:
an ultrasonic probe configured to transmit an ultrasonic wave to a subject, receive an ultrasonic wave reflected by the subject, and generate an echo signal corresponding to the received ultrasonic wave;
a generation unit configured to generate an ultrasonic image associated with the subject based on the generated echo signal;
a calculation unit configured to calculate edge information of each of a plurality of pixels included in the generated ultrasonic image based on spatial derivation of a brightness value of said each pixel;
a filter processing unit configured to generate a filtered image from the ultrasonic image by applying a filter having a filter characteristic corresponding to the calculated edge information to the ultrasonic image;
a compositing unit configured to generate a composite image of the generated filtered image and the ultrasonic image in accordance with a compositing ratio corresponding to a brightness value of the filtered image; and
an enhancement unit configured to generate an enhanced image from the generated composite image by increasing a brightness value of each of a plurality of pixels included in the composite image in accordance with the edge information.

12. An ultrasonic diagnostic apparatus comprising:
an ultrasonic probe configured to transmit an ultrasonic wave to a subject, receive an ultrasonic wave reflected by the subject, and generate an echo signal corresponding to the received ultrasonic wave;
a generation unit configured to generate an ultrasonic image associated with the subject based on the generated echo signal;
a table application unit configured to generate a table image from the generated enhanced image by applying a table to the enhanced image, the table having a first input/output characteristic in which an output brightness value linearly increases with an increase in input brightness value in a brightness value range smaller than a threshold, and a second input/output characteristic in which an output brightness value nonlinearly increases more gradually than in the first input/output characteristic with an increase in input brightness value in a brightness value range larger than the threshold;
a calculation unit configured to calculate edge information of each of a plurality of pixels included in the generated table image or the ultrasonic image based on spatial derivation of a brightness value of said each pixel;
a filter unit configured to generate a filtered image from the table image by applying a filter having a filter characteristic corresponding to the calculated edge information to the table image; and
an enhancement unit configured to generate an enhanced image from the generated filtered image by increasing a brightness value of each of a plurality of pixels included in the filtered image in accordance with the edge information.

13. An image processing apparatus comprising:
a storage unit configured to store data of a medical image associated with a subject;
a calculation unit configured to calculate edge information based on the medical image;
a filter unit configured to generate a filtered image from the medical image by performing a filter having a filter characteristic corresponding to the calculated edge information to the medical image;
an enhancement unit configured to generate an enhanced image from the generated filtered image by increasing a brightness value of a portion, of the filtered image, which corresponds to the edge information in accordance with the edge information; and
a compositing unit configured to generate a composite image of the generated enhanced image and the medical image in accordance with a compositing ratio corresponding to a brightness value of the enhanced image.

14. An image processing apparatus comprising:
a storage unit configured to store data of a medical image associated with a subject;
a calculation unit configured to calculate edge information of each of a plurality of pixels included in the medical image based on spatial derivation of a brightness value of said each pixel;
a filter processing unit configured to generate a filtered image from the medical image by performing a filter having a filter characteristic corresponding to the calculated edge information to the medical image;
an enhancement unit configured to generate an enhanced image from the generated filtered image by increasing a brightness value of each of a plurality of pixels included in the filtered image in accordance with the edge information; and
a table application unit configured to generate a table image from the generated enhanced image by applying a table to the enhanced image, the table having a first input/output characteristic in which an output brightness value linearly increases with an increase in input brightness value in a brightness value range smaller than a threshold, and a second input/output characteristic in which an output brightness value nonlinearly increases more gradually than in the first input/output characteristic with an increase in input brightness value in a brightness value range larger than the threshold.

15. An image processing apparatus comprising:
a storage unit configured to store data of a medical image associated with a subject;
a calculation unit configured to calculate edge information of each of a plurality of pixels included in the medical image based on spatial derivation of a brightness value of said each pixel;
a filter unit configured to generate a filtered image from the medical image by performing a filter having a filter characteristic corresponding to the calculated edge information to the medical image;
a compositing unit configured to generate a composite image of the generated filtered image and the medical image in accordance with a compositing ratio corresponding to a brightness value of the filtered image; and
an enhancement unit configured to generate an enhanced image from the generated composite image by increasing a brightness value of each of a plurality of pixels included in the composite image in accordance with the edge information.

16. An image processing apparatus comprising:
a storage unit configured to store data of a medical image associated with a subject;
a table application unit configured to generate a table image from the medical image by applying a table to the medical image, the table having a first input/output characteristic in which an output brightness value linearly increases with an increase in input brightness value in a brightness value range smaller than a threshold, and a second input/output characteristic in which an output brightness value nonlinearly increases more gradually than in the first input/output characteristic with an increase in input brightness value in a brightness value range larger than the threshold;
a calculation unit configured to calculate edge information of each of a plurality of pixels included in the generated table image or the medical image based on spatial derivation of a brightness value of said each pixel;
a filter unit configured to generate a filtered image from the table image by applying a filter having a filter characteristic corresponding to the calculated edge information to the table image; and
an enhancement unit configured to generate an enhanced image from the generated filtered image by increasing a brightness value of each of a plurality of pixels included in the filtered image in accordance with the edge information.
